(19) United States
(12) Patent Application Publication
(10) Pub. No.: US 2020/0282100 A1
(43) Pub. Date: Sep. 10, 2020

(54) Hemostatic Dressings with Self-Assembling Peptide Hydrogels

(71) Applicant: 3-D Matrix, Ltd., Tokyo (JP)

(72) Inventors: Eun Seok Gil, Lexington, MA (US); Elton Aleksi, West Roxbury, MA (US); Marc Rioult, Walpole, MA (US)

(21) Appl. No.: 16/304,968

(22) PCT Filed: Jun. 1, 2017
(86) PCT No.: PCT/US2017/035424

§ 371 Date: Nov. 27, 2018

Related U.S. Application Data
(60) Provisional application No. 62/344,176, filed on Jun. 1, 2016.

(51) Int. Cl. A61L 15/26; A61L 15/28; A61L 15/42; A61L 15/44; A61L 15/64; A61L 15/32

(52) U.S. Cl. CPC A61L 15/26; A61L 15/28; A61L 15/32; A61L 15/44; A61L 15/64; A61L 15/425

(57) ABSTRACT

Hemostatic dressings are synergistically used in conjunction with self-assembling peptide hydrogels to promote hemostasis at a target site. Related methods, kits, and devices for hemostasis are disclosed.

### FIELD OF THE TECHNOLOGY

One or more aspects relate to hemostatic dressings used in conjunction with self-assembling peptide hydrogels for various medical, research, and industrial applications.

### BACKGROUND

Hemostasis generally relates to the prevention of blood loss from vessels and organs of the body of a subject. The process plays an important role in stopping or otherwise controlling blood flow during surgery, medical treatment, and wound healing. While hemostasis is a natural biological process involving coagulation, various chemical, mechanical, and physical agents may be implemented to achieve or promote hemostasis.

### SUMMARY

In accordance with one or more aspects, a kit for hemostasis may comprise a wound dressing, and a solution comprising a self-assembling peptide comprising between about 7 amino acids and 32 amino acids in an effective amount and in an effective concentration for use in forming a hydrogel under physiological conditions to promote hemostasis.

In some aspects, the self-assembling peptide may be selected from the group consisting of RADA16, IEIK13, and KLD12. The self-assembling peptide may comprise between about 12 to about 16 amino acids that alternate between a hydrophobic amino acid and a hydrophilic amino acid. The solution may be substantially free of cells and/or drugs.

In some aspects, the wound dressing may be substantially porous. The wound dressing may comprise a sponge, a woven textile, a non-woven textile, a puff, or a mixture thereof. The wound dressing may comprise cotton gauze. The wound dressing may comprise a synthetic material. The wound dressing may comprise a bio-absorbable material. In at least some non-limiting aspects, the wound dressing may comprise collagen, gelatin, chitosan, hyaluronic acid, starch, silk fibroin, oxidized regenerated cellulose, homopolymers of lactide or glycolide, or copolymers of lactide and glycolide.

In some aspects, the solution may be provided in a volume of about 1 μL to 2 mL per 1 cm² of wound dressing surface. The kit may further comprise instructions for administering the wound dressing and the solution to a target area for hemostasis. The instructions may involve applying tactile pressure to a top of the wound dressing at the target area. In at least some non-limiting aspects, the kit may further comprise at least one of a syringe, a bottle, a transfer tool, a spreader, and a container.

In accordance with one or more aspects, a device for hemostasis may comprise a porous wound dressing, and a solution comprising a self-assembling peptide pervading the pores of the porous wound dressing, the self-assembling peptide comprising between about 7 amino acids and 32 amino acids in an effective amount and in an effective concentration for use in forming a hydrogel under physiological conditions to promote hemostasis.

In some aspects, the solution may be present in a volume of about 1 μL to 2 mL per 1 cm² of porous wound dressing.

In some embodiments, the kit and/or device may provide hemostasis to a target area having a bleeding score of 2 or more on the WHO Bleeding Scale. In some embodiments, the kit and/or device may provide hemostasis to a target area in 2 minutes or less. Specifically, the kit and/or device may reduce a bleeding score of a target area to 0 on the WHO Bleeding Scale in 2 minutes or less. In some embodiments, the kit and/or device may provide hemostasis to a target area having an initial bleeding score of 3 or 4 on the WHO Bleeding Scale in 2 minutes or less, upon applying tactile pressure to a top of a wound dressing at the target area.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. The accompanying drawings are included to provide illustration and a further understanding of the various aspects and embodiments, and are incorporated in and constitute a part of this specification. The drawings, together with the remainder of the specification, serve to explain principles and operations of the described and claimed aspects and embodiments.

### BRIEF DESCRIPTION OF THE DRAWINGS

The accompanying drawings are not intended to be drawn to scale. For purposes of clarity, not every component may be labeled. In the drawings:

FIG. 1 includes six images of a process for using a hemostatic porous dressing with a self-assembling peptide hydrogel, according to one embodiment;

FIG. 2 includes six images of an alternate process for using a hemostatic porous dressing with a self-assembling peptide hydrogel, according to another embodiment;

FIG. 3 includes four images of the use of a hemostatic porous dressing with self-assembling peptide hydrogels, according to one embodiment;

FIG. 4 is a visualization of gel formation in conjunction with various hemostatic porous dressings, according to embodiments described herein;

FIG. 5 is a visualization of gel formation in conjunction with a hemostatic porous sponge, according to one embodiment;

FIG. 6 includes two images of wound defect sites treated with a hemostatic sponge, according to embodiments described herein;

FIG. 7 is a graph of the degree of bleeding (bleeding score) over time of samples treated with a hemostatic sponge and saline and samples treated with a hemostatic sponge and a self-assembling peptide hydrogel; and

FIG. 8 is a graph of hemostatic success (%) over time achieved in the samples treated with a hemostatic sponge and saline and the samples treated with a hemostatic sponge and a self-assembling peptide hydrogel.

### DETAILED DESCRIPTION

In accordance with one or more embodiments, self-assembling peptide hydrogels may be used as a scaffold for hemostasis. PuraMatrix® peptide hydrogel (hereinafter “PuraMatrix®”), commercially available from 3-D Matrix Co., Ltd., for example, is a synthetic, 16-amino acid polypeptide with a repeating sequence of arginine, alanine, and aspartic acid, or RADARADARADARADA (RADA16). PuraMatrix® is known to self-assemble to form a hydrogel under physiological conditions and can be used for various biomedical applications. In accordance with various embodiments described herein, PuraMatrix® may be used for hemostasis. Other relevant non-limiting synthetic peptide sequences may be represented by self-assembling peptides having the repeating sequence of lysine, leucine, and aspartic acid (Lys-Leu-Asp (KLD)), and such peptide sequences are represented by (KLD)p, wherein p=2-50, such as KLD12. Still other relevant non-limiting synthetic peptide sequences may be represented by self-assembling peptides having the repeating sequence of isoleucine, glutamic acid, isoleucine and lysine (Ile-Glu-Ile-Lys (IEIK)), and such peptide sequences are represented by (IEIK)p, wherein p=2-50, such as IEIK13. Other embodiments may involve still other self-assembling peptides. In some non-limiting embodiments, peptide hydrogels such as those disclosed in International Patent Application Publication No. WO2015/138514 titled “SELF-ASSEMBLING PEPTIDE COMPOSITIONS” and assigned to 3-D Matrix, Ltd., which is hereby incorporated herein by reference in its entirety for all purposes, may be implemented.

Embodiments disclosed herein may comprise certain peptide compositions (and particularly certain compositions of self-assembling peptide agents), and technologies relating thereto. In some embodiments, such compositions may be or comprise solutions. In some embodiments, such compositions may be or comprise gels. In some embodiments, such compositions may be or comprise solid (e.g., dried/lyophilized) peptides. For example, particular peptide compositions (i.e., peptide compositions having specific concentration, ionic strength, pH, viscosity and/or other characteristics) have useful and/or surprising attributes (e.g., gelation or self-assembly kinetics [e.g., rate of gelation and/or rate and reversibility of peptide self-assembly], stiffness [e.g., as assessed via storage modulus], and/or other mechanical properties).

In some embodiments, peptides included in provided compositions are self-assembling peptides. In some embodiments, peptides included in provided compositions are amphiphilic peptides. In some embodiments, peptides included in provided compositions have an amino acid sequence characterized by at least one stretch (e.g., of at least 4, 5, 6, 7, 8, 9, 10, 11, 12, 13, 14, 15, 16, 17, 18, 19, 20 etc amino acids) of alternating hydrophilic and hydrophobic amino acids. In accordance with one or more embodiments, peptide compositions may include an amphiphilic polypeptide having about 6 to about 200 amino acid residues. In some embodiments, a peptide may have a length within the range of about 6 to about 20 amino acids and an amino acid sequence of alternating hydrophobic amino acid and hydrophilic amino acids.

In some embodiments, peptides included in provided compositions have an amino acid sequence that includes one or more repeats of Arg-Ala-Asp-Ala (RADA). In some embodiments, peptides included in provided compositions have an amino acid sequence that comprises or consists of repeated units of the sequence Lys-Leu-Asp-Leu (KLDL). In some embodiments, peptides included in provided compositions have an amino acid sequence that comprises or consists of repeated units of the sequence Ile-Glu-Ile-Lys (IEIK). In some embodiments, the peptides may be IEIK13, KLD12, or RADA16. In some embodiments, compositions of these peptides may have enhanced properties relative to appropriate reference compositions that have different (e.g., lower) pH level, and/or ionic strength.

In some embodiments, increased ionic strength may beneficially impact stiffness and/or gelation kinetics to peptide compositions rendering them suitable for a broader range of applications. In some embodiments, increased ionic strength may be physiological ionic strength, which may occur when peptide compositions are placed into the body. In some embodiments, an ionic strength of a peptide composition may be about 0.0001 M to about 1.5 M. In some embodiments, an ionic strength of a peptide composition may be adjusted by mixing common salts, for example, NaCl, KCl, MgCl₂, CaCl₂, CaSO₄, DPBS (Dulbecco's Phosphate-Buffered Saline, 10×). In some embodiments, ionic strengths of peptide compositions may be adjusted by mixing common salts, wherein one or more common salts are composed of one or more salt forming cations and one or more salt forming anions, wherein the salt forming cations are selected from the group consisting of ammonium, calcium, iron, magnesium, potassium, pyridinium, quaternary ammonium, and sodium, wherein the salt forming anions are selected from the group consisting of acetate, carbonate, chloride, citrate, cyanide, fluoride, nitrate, nitrite, and phosphate.

In accordance with one or more aspects, properties of certain peptide compositions, including but not limited to IEIK13, KLD12, and RADA16, may be enhanced by maintaining their pH level at about 3.5 or less and, at the same time, their salt concentrations at less than their critical ionic strength levels (i.e. no precipitation). In some embodiments, a peptide composition may have a pH within the range of about 2.5 to about 4.0, or within the range of about 3.0 to about 4.0. In some embodiments, provided compositions have a pH at or above about 2.5, 2.6, 2.7, 2.8, 2.9, 3.0, about 3.1, about 3.2, about 3.3, about 3.4, about 3.5 or higher. In some embodiments, provided compositions have a pH at or below about 4.3, about 4.2, about 4.1, about 4.0, about 3.9, about 3.7, about 3.6, about 3.5, about 3.4, or lower. In some embodiments, pH of a peptide composition can be achieved with a solution selected from the group consisting of sodium hydroxide or, potassium hydroxide, calcium hydroxide, sodium carbonate, sodium acetate, sodium sulfide, DMEM (Dulbecco's modified Eagle's medium), and PBS (Phosphate-Buffered Saline).

In some embodiments, a peptide composition may be solution, gel, or any combination thereof. In some embodiments, peptide concentration in a peptide composition is at least 0.05%, at least 0.25%, at least 0.5%, at least 0.75%, at least 1.0% or more. In some embodiments, peptide concentration in a peptide composition is less than 5%, less than 4.5%, less than 4%, less than 3.5%, less than 3%, or less. In some embodiments, peptide concentration in a peptide composition is within a range between about 0.5% and about 3%. In some embodiments, peptide concentration in a peptide composition is within a range between about 0.5% and about 2.5%. In some embodiments, peptide concentration in a peptide composition is within a range between about 1% and about 3%. In some embodiments, peptide concentration in a peptide composition is within a range between about 1% and about 2.5%. In some embodiments, peptide concentration in a peptide composition is about 0.5%, about 1%, about 1.5%, about 2%, about 2.5%, about 3%, or more. In some particular embodiments, where the peptide is RADA16, peptide concentration in peptide compositions is within a range of about 0.05% to about 10%.

In some embodiments, a peptide composition may have a viscosity with the range of about 1 to about 10000 Pa-S. In some embodiments, a peptide composition may have a storage modulus with the range of about 50 to about 2500 Pa.

The term “peptide” as used herein refers to a polypeptide that is typically relatively short, for example, having a length of less than about 100 amino acids, less than about 50 amino acids, less than 20 amino acids, or less than 10 amino acids.

The term “polypeptide” as used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide.

In some embodiments, the term “polypeptide” may be appended to a name of a reference polypeptide, activity, or structure. In such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known. In some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class. In some embodiments, a member of a polypeptide class or family shows significant sequence homology or identity, shares a common sequence motif, and/or shares a common activity with all polypeptides within the class.

For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90%> or even 95%, 96%, 97%, 98%, or 99%. Such a conserved region usually encompasses at least 3-4 and often up to 20 or more amino acids. In some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

The term “self-assembling” is used herein in reference to certain polypeptides that, under appropriate conditions, can spontaneously self-associate into structures. For example, such that solutions (e.g., aqueous solutions) containing them develop gel character. In some embodiments, interactions between and among individual self-assembling polypeptides within a composition are reversible, such that the composition may reversibly transition between a gel state and a solution state. In some embodiments, self-assembly (and/or disassembly) is responsive to one or more environmental triggers (e.g., change in one or more of pH, temperature, ionic strength, osmolarity, osmolality, applied pressure, applied shear stress, etc). In some embodiments, compositions of self-assembling polypeptides are characterized by detectable beta-sheet structure when the polypeptides are in an assembled state.

In accordance with one or more embodiments, self-assembling peptide hydrogels may be used with a hemostatic dressing as a scaffold for hemostasis. In accordance with one or more aspects, the hemostatic properties of various hemostatic dressings may be enhanced by using them in conjunction with self-assembling peptide hydrogels. In accordance with one or more further aspects, the hemostatic properties of self-assembling peptide hydrogels may be enhanced by using them in conjunction with various hemostatic dressings. Various embodiments described herein are therefore directed to the synergy exhibited by concurrent use of hemostatic dressings and self-assembling peptide hydrogels for hemostasis.

In some embodiments disclosed herein, self-assembling peptide hydrogels used with hemostatic dressings may provide hemostasis to a target area experiencing heavy bleeding, upon applying tactile pressure to a top of a wound dressing at the target area.

Hemostasis is the first stage of wound healing. As disclosed herein “hemostasis” is used to reference a reduction in bleeding. For example, hemostasis may refer to a reduction in bleeding of an open wound. In some embodiments, hemostasis is defined as a complete stop in bleeding. In some embodiments, hemostasis is defined as a significant stop in bleeding. Generally, hemostasis refers to a visually significant reduction in bleeding of an open wound.

In accordance with certain embodiments, the self-assembling peptide hydrogels used with hemostatic dressings, as disclosed herein, may be used to stop heavy bleeding. For instance, embodiments disclosed herein may stop bleeding of a scale of 2 or higher on the World Health Organization (WHO) Bleeding Scale. The WHO Bleeding Scale is a clinical investigator-assessed five-point scale with 0=No bleeding, 1=Petechiae, 2=Mild blood loss, 3=Gross blood loss, and 4=Debilitating blood loss. Embodiments disclosed herein may be used to treat wounds classified as producing mild blood loss (2 on the WHO scale), gross blood loss (3 on the WHO scale), or debilitating blood loss (4 on the WHO scale). In accordance with certain embodiments, hemostasis is achieved when bleeding is a 1 or lower on the WHO scale. For instance, hemostasis may be achieved when bleeding is visually determined to be a 1, 0.5, or 0 on the WHO bleeding scale. For instance, in some embodiments disclosed herein, self-assembling peptide hydrogels used with hemostatic dressings may reduce bleeding of a target area to a bleeding score of 0.5 or less on the WHO Bleeding Scale, upon applying tactile pressure to a top of a wound dressing at the target area. Self-assembling peptide hydrogels used with hemostatic dressings may reduce bleeding of a target area to a bleeding score of 0 on the WHO Bleeding Scale, for example after 2 minutes of applying tactile pressure to the target area.

In accordance with one or more non-limiting embodiments, the self-assembling peptide hydrogel may be IEIK13, KLD12, or RADA16. The self-assembling peptide may comprise between about 7 amino acids and 32 amino acids in an effective amount and in an effective concentration for use in forming a hydrogel under physiological conditions to promote hemostasis. In some specific embodiments, the self-assembling peptide may comprise between about 12 to about 16 amino acids that alternate between a hydrophobic amino acid and a hydrophilic amino acid. The peptide hydrogel may gel upon contact with blood to stop and/or control bleeding via mechanical blocking of a bleeding site. Upon gelation, a resulting peptide hydrogel may be substantially transparent so as to allow unobstructed viewing of a target area. The peptide hydrogels may generally be characterized as non-biogenic, biocompatible, and resorbable. The self-assembling peptide hydrogel may be present in solution at varying concentrations. For example, in some non-limiting embodiments, a 2.5% peptide hydrogel solution may be used. In at least some embodiments, the solution may be substantially free of cells and/or drugs. In other embodiments, the solution may include one or more therapeutic agents to promote hemostasis. As described further herein, the solution may be formulated, such as to impact its stiffness and/or gelation kinetics, or to provide a suitable environment for an intended application.

Generally, self-assembling peptide hydrogels alone may be used to treat bleeding of a scale of 1 or less on the WHO Bleeding Scale. When directly applied to a wound or treatment site, a self-assembling peptide hydrogel, substantially free of agents, and used without a dressing may not be effective at achieving hemostasis of a heavy bleeding wound site. For instance, a self-assembling peptide hydrogel, with nothing more, may not stop heaving bleeding of a scale of 3 or 4 on the WHO Bleeding Scale. Accordingly, while self-assembling peptide hydrogels may be used as a scaffold for hemostasis, and may be capable of achieving hemostasis of certain wounds, the peptide hydrogels, generally, may not achieve hemostasis of wounds classified as having gross or debilitating blood loss (3 or 4 on the WHO scale). Embodiments disclosed herein, which combine self-assembling peptide hydrogels and a wound dressing, may synergistically achieve hemostasis of wounds having blood loss of a 2 or greater on the WHO Bleeding Scale.

In accordance with one or more embodiments, a target pH level and/or tonicity level for the solution may be selected at least in part based on the type of cell or tissue involved in an intended application. For example, a pH level of the peptide hydrogel may be adjusted to a level of up to about 3.5, for example, up to a level of about 3.4, for improved cell viability by providing a more gentle, less harsh environment. With respect to tonicity, the tonicity of a peptide hydrogel solution may be adjusted so as to closely match the plasma osmolality of a target cell type and/or target species. For example, the tonicity of the peptide hydrogel solution may be adjusted based on the plasma osmolality of any given cell type. Tonicity levels may range depending on the type of species and/or the type of cell or tissue involved. In some non-limiting embodiments, a target tonicity may range from about 260 to about 360 mOsm/L.

Generally, a number of therapeutic sites may be treated as described herein. A therapeutic site may refer to a site of injury. Therapeutic sites may be exterior or interior sites. Exterior therapeutic sites include superficial and/or exterior bleeding sites or open wounds experiencing blood loss of a scale of 2 or higher on the WHO Bleeding Scale. Exterior therapeutic sites may include sites of trauma or amputation. Interior sites may include surgical incisions made on exposed tissues experiencing a blood loss of a scale of 2 or higher on the WHO bleeding scale. Interior sites may include surgical incisions for the purpose of surgical treatment, or internal bleeding sites that have been at least partially exposed for treatment. In some embodiments, interior sites include therapeutic sites treated by endoscopic and/or laparoscopic procedures.

In accordance with one or more embodiments, the hemostatic dressing may have a dressing surface generally corresponding in size and/or shape to a therapeutic target site. The hemostatic dressing may be configured to substantially cover a therapeutic target site. The hemostatic dressing may be porous. In some embodiments, the wound dressing may be in the form of a sponge, a woven textile, a non-woven textile, a puff, or a mixture thereof. In some specific embodiments, the wound dressing may be made of cotton gauze. In some embodiments, the wound dressing may instead be made of a synthetic material. In at least some embodiments, the wound dressing may be surgical grade. For instance, the wound dressing may be made of a bio-absorbable, bio-compatible, or sterile material. The wound dressing may include collagen, gelatin, chitosan, hyaluronic acid, starch, silk fibroin, oxidized regenerated cellulose, homopolymers of lactide or glycolide, and/or copolymers of lactide and glycolide. In some specific non-limiting embodiments, the hemostatic porous dressing may be Surgifoam® or Surgicel® hemostatic dressings both commercially available from Ethicon, Gelfoam® hemostatic dressing commercially available from Pfizer, or Helistat® hemostatic dressing commercially available from Integra.

Hemostatic dressings are generally capable of stopping heavy blood flow from large wounds. For instance, when applied with pressure, hemostatic dressings may stop hemorrhage from large arteries and veins within several minutes of application. Hemostatic dressings disclosed herein, when applied without a self-assembling peptide hydrogel, may achieve hemostasis from a heavily bleeding wound (3 or 4 on the WHO scale) in about 5 to about 8 minutes. When used with a self-assembling peptide hydrogel, as described herein, hemostatic dressings and hydrogels may achieve hemostasis from a similar heavily bleeding wound in about 5 minutes or less. Specifically, embodiments disclosed herein may provide hemostasis to a target area having a bleeding score of 3 or 4 on the WHO Bleeding Scale in 2 minutes or less. Generally, hemostatic dressings and self-assembling peptides may be applied to the target area concurrently with tactile pressure to the top of the hemostatic dressing.

As noted above, the peptide hydrogel and the hemostatic dressing may be used in conjunction in accordance with various embodiments. This combination may beneficially impart relatively fast and easy delivery of the peptide hydrogel solution to a target location, such as a wound area or a surgical site, in comparison to alternative approaches such as those involving sole application. This combination may also beneficially impart assistance with respect to the application of hand or finger pressure, which can be applied on the top of the porous dressing to temporarily hold bleeding flow which, in turn, may achieve stable gelation of the self-assembling peptide hydrogel near the bleeding wound surface without hindrance by the bleeding flow. The combination may also beneficially provide a reservoir space in the porous dressing which may contain peptide solution so as to allow for the release of reserved peptide solution onto the wound when it is squeezed by a hand or finger. Peptide hydrogel may at the same time be retained in the reservoir space to cover a target area. The viscosity of the peptide hydrogel solution may also beneficially impart a sticky property which may cause the hemostatic dressing to more stably stay in position on a target area.

In accordance with one or more embodiments, the peptide hydrogel solution may be provided in a volume of about 1 μL to 2 mL per 1 cm² of wound dressing surface. For instance, the solution may be provided in a volume of about 1 μL, 2 μL, 5 μL, 10 μL, 50 μL, 100 μL, 200 μL, 500 μL, 750 μL, 1 mL, 1.25 mL, 1.5 mL, 1.75 mL, or 2 mL. The peptide hydrogel solution may be provided in a volume exceeding the volume requirement for the wound dressing surface. For instance, administering the dressing and peptide hydrogel to a surface may comprise administering or infusing an excess volume of peptide hydrogel onto the dressing and removing excess hydrogel prior to application of the infused dressing on the wound site.

In accordance with one or more embodiments, a hemostatic dressing and peptide solution may be combined together in a single device. The device may include a porous wound dressing and a solution comprising a self-assembling peptide pervading the pores of the porous wound dressing. The self-assembling peptide may comprise between about 7 amino acids and 32 amino acids in an effective amount and in an effective concentration for use in forming a hydrogel under physiological conditions to promote hemostasis. The device may be prepackaged for use at a target area. The packaging may include instructions for administering the device to a target area for hemostasis. The instructions may further involve direction to apply tactile pressure to a top of the device at the target area. In at least some embodiments, the device may be surgical grade. For instance, the device may be made of a bio-absorbable, bio-compatible, or sterile material.

In accordance with one or more other embodiments, a kit for hemostasis may be provided. The kit may include both a hemostatic dressing and a peptide hydrogel solution. The two components may be packaged together along with instructions for use. The instructions may provide guidance for how to introduce the peptide hydrogel solution to the hemostatic dressing prior to or during use in connection with a target area. The kit may include one or more further components to facilitate the combination of the hemostatic dressing and the peptide hydrogel solution prior to or during use. For example, such components may include a syringe for injecting the self-assembling peptide hydrogel solution onto the hemostatic dressing surface. Other components may include a transferring device to transfer the peptide solution from a bottle to the dressing, such as a pipette or scoop. In still other embodiments, the kit may include a stick to uniformly spread the peptide solution on the dressing surface and/or a container dish to hold the dressing during the spreading process. The kit may include instructions for administering the hemostatic dressing and peptide hydrogel to a target area for hemostasis. The instructions may further involve direction to apply tactile pressure to a top of the dressing at the target area.

In still other embodiments, a hemostatic dressing and a peptide hydrogel solution may be packaged and provided separately from each other. Each may be packaged as a separate product and then combined prior to or during use. One or both separately packaged components may include instructions for administering the hemostatic dressing and peptide hydrogel to a target area for hemostasis. The instructions may further involve direction to apply tactile pressure to a top of the dressing at the target area. One or both separately packaged components may also optionally include additional components such as those described above to facilitate the concurrent usage.

The function and advantages of these and other embodiments will be more fully understood from the following non-limiting examples. The examples are intended to be illustrative in nature and are not to be considered as limiting the scope of the embodiments discussed herein.

### EXAMPLES

### Example 1

The following example illustrates the use of self-assembling peptide hydrogels with various hemostatic porous dressings.

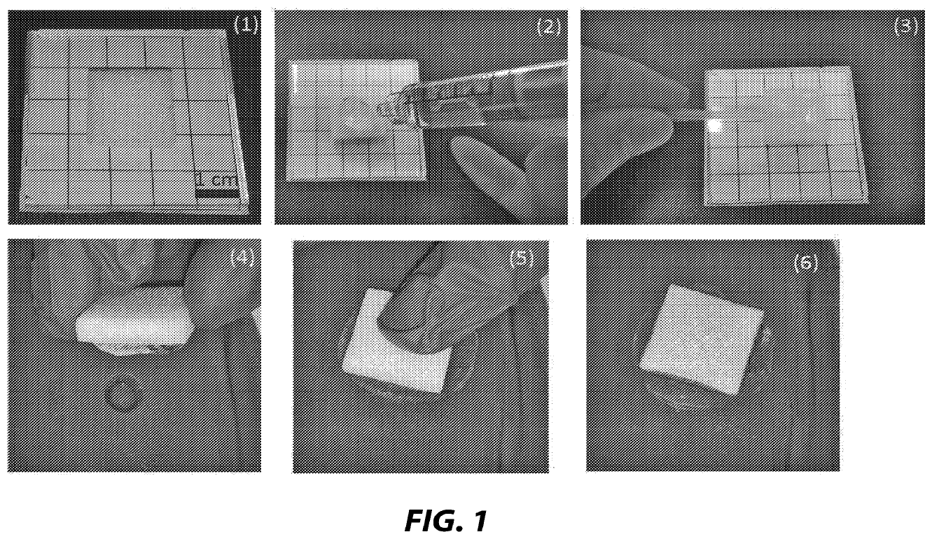

FIG. 1 provides an overview of a process for using a hemostatic porous dressing with a self-assembling peptide hydrogel. In (1), an absorbable gelatin sponge (Surgifoam®, Ethicon) (2 cm×2 cm×0.7 cm (4 cm²)) is provided. In (2), RADA16 2.5% (PuraMatrix®) (1 mL) is injected. In (3), RADA16 2.5% is spread on the surface of the gelatin sponge. In (4), bleeding is observed in association with a wound, blood is removed from the wound, and the hemostatic porous dressing with the self-assembling peptide hydrogel is applied to the wound. In (5), the dressing on the wound is covered and the sponge is pushed with the hand or finger until hemostasis is achieved. In (6), hemostasis is achieved.

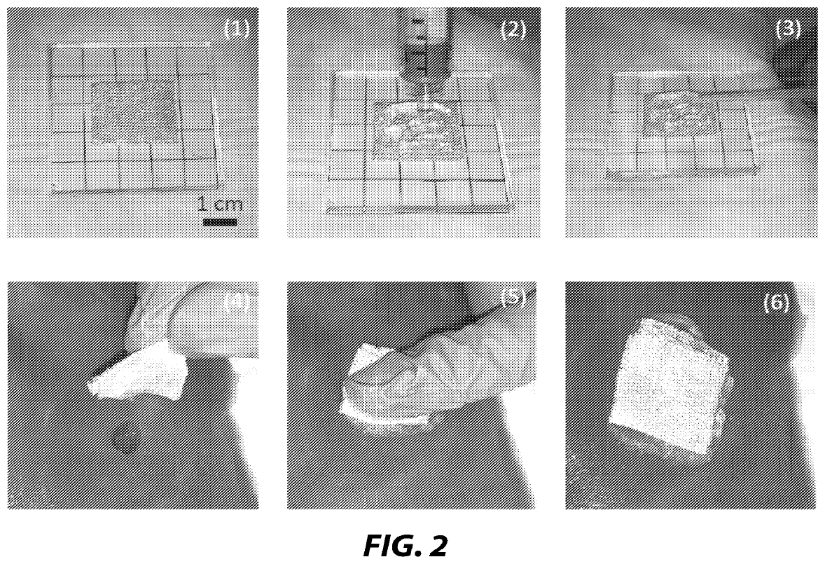

FIG. 2 provides an overview of another process for using a hemostatic porous dressing with a self-assembling peptide hydrogel. In (1), an absorbable oxidized regenerated cellulose woven dressing (Surgicel®, Ethicon) (2.5 cm×2 cm (5 cm²)) is provided. In (2), RADA16 2.5% (PuraMatrix®) (1 mL) is injected. In (3), RADA16 2.5% is spread on the surface of the gelatin sponge. In (4), bleeding is observed in association with a wound, blood is removed from the wound, and the hemostatic porous dressing with the self-assembling peptide hydrogel is applied to the wound. In (5), the dressing on the wound is covered and the sponge is pushed with the hand or finger until hemostasis is achieved. In (6), hemostasis is achieved.

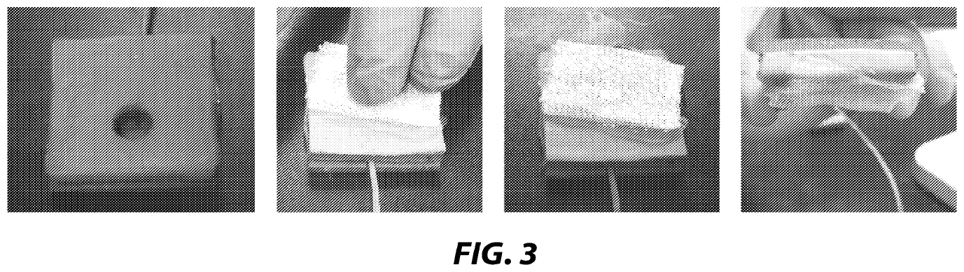

FIG. 3 further illustrates the use of a hemostatic porous dressing with self-assembling peptide hydrogels. In (1), an absorbable gelatin sponge (Surgifoam®, Ethicon) (2 cm×2 cm×0.7 cm (4 cm²)) with RADA16 2.5% (1 mL) is applied on an in vitro wound model. In (2), an absorbable oxidized regenerated cellulose woven dressing (Surgicel®, Ethicon) (2.5 cm×2 cm (5 cm²)) with RADA16 2.5% (1 mL) is applied on an in vitro wound model.

### Example 2

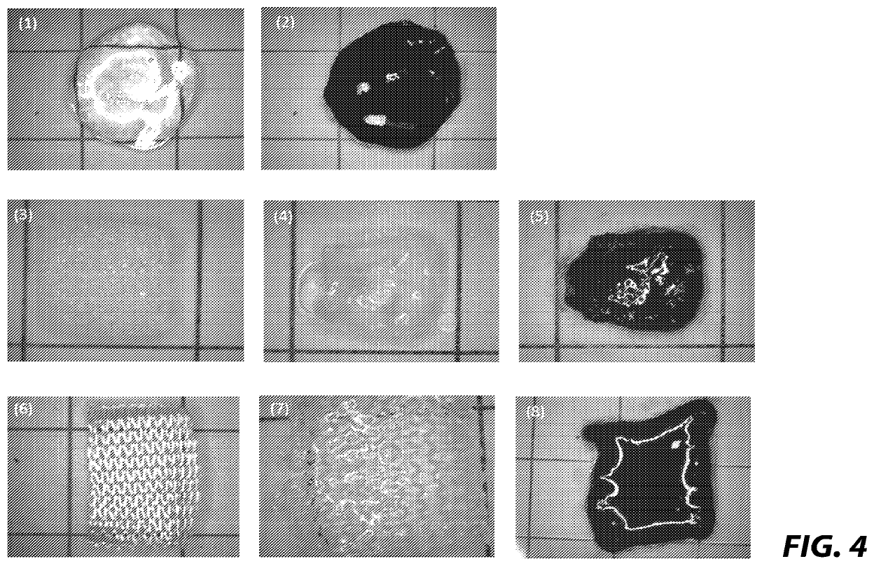

The capability of a peptide hydrogel to gelate when used in conjunction with a hemostatic porous dressing was demonstrated. A Congo Red assay was performed to assess gel formation of peptide solutions in a PBS buffer solution (pH 7.4) when used with a hemostatic porous dressing. RADA16 2.5% (PuraMatrix®) was plated on a glass slide and also on a hemostatic porous dressing on a glass slide. PuraMatrix® was spread on the surface of the hemostatic porous dressing using a stick. After 30 seconds, 1% Congo Red solution was added around and on top of the gel aliquots and then the excess Congo Red solution was wiped off prior to examination. RADA16 2.5% was also plated on an absorbable gelatin sponge (Surgifoam®, Ethicon) and on an absorbable oxidized regenerated cellulose woven dressing (Surgicel®, Ethicon). Visualization of gel formation determined the success or failure of gelation. As shown in FIG. 4, RADA16 2.5% gelled in conjunction with the various hemostatic porous dressings similar to the extent observed in pure RADA16 2.5%. Specifically, (1) and (2) show pure RADA16 2.5% (PuraMatrix®). In (3), (4), and (5), RADA16 2.5% is shown with absorbable gelatin sponge (Surgifoam®, Ethicon). In (6), (7), and (8), RADA16 2.5% is shown with absorbable oxidized regenerated cellulose woven dressing (Surgicel®, Ethicon).

Accordingly, as shown in FIG. 4, RADA16 2.5% is capable of gelation when used with an absorbable gelatin sponge and with an absorbable oxidized regenerated cellulose woven dressing. The gelated self-assembling peptide and dressing combination may be capable of promoting hemostasis on a bleeding wound.

### Example 3

The capability of a peptide hydrogel to gelate when used in conjunction with a hemostatic porous dressing was demonstrated. A Congo Red assay was performed to assess gel formation of peptide solutions in a PBS buffer solution (pH 7.4) when used with a hemostatic porous dressing.

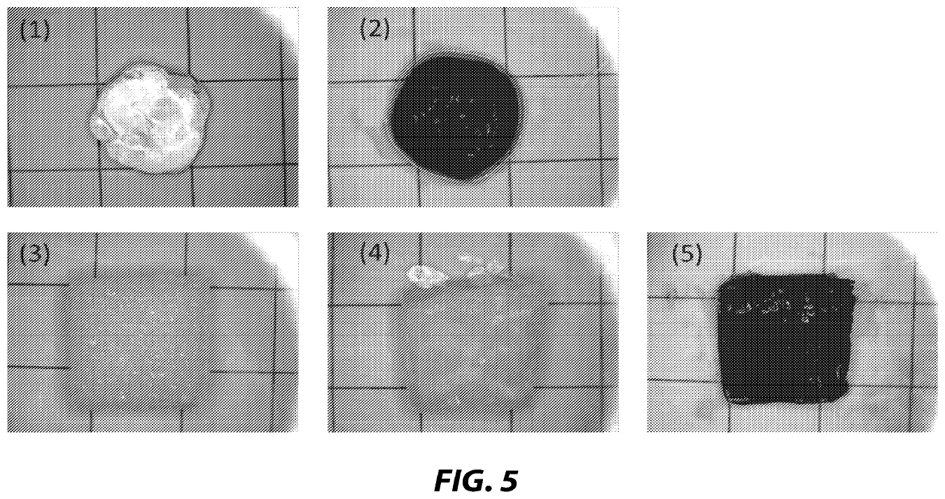

IEIK13 1.3% (pH 3.0) was plated on a glass slide and on a hemostatic porous dressing on a glass slide. IEIK13 1.3% (pH3.0) was spread on the surface of hemostatic porous dressing using a stick. After 30 seconds, 1% Congo Red solution was added around and on top of the gel aliquots and then the excess Congo Red solution was wiped off prior to examination. IEIK13 1.3% (pH 3.0) was also plated on absorbable gelatin sponge (Gelfoam®, Pfizer). Visualization of gel formation determined the success or failure of gelation. As shown in FIG. 5, IEIK13 1.3% (pH 3.0) gelled with hemostatic porous dressing similar to the extent observed in pure IEIK13 1.3% (pH 3.0). Specifically, (1) and (2) show pure IEIK13 1.3% (pH 3.0). In (3), (4), and (5), IEIK13 1.3% (pH 3.0) is shown with absorbable gelatin sponge (GelFoam®, Pfizer).

Accordingly, as shown in FIG. 5, IEIK13 1.3% (pH 3.0) is capable of gelation when used with an absorbable gelatin sponge. It is expected that similar results would be obtained for IEIK13 1.3% (pH 3.0) on an absorbable oxidized regenerated cellulose woven dressing, as observed with RADA16 2.5%. The gelated self-assembling peptide and dressing combination may be capable of promoting hemostasis on a bleeding wound.

### Example 4

The following comparative example illustrates the enhanced hemostatic efficacy of a hemostatic sponge when utilized with a self-assembling peptide hydrogel.

A study was performed to evaluate the efficacy of hemostatic agents in an organ wounding model in swine. A midline laparotomy was performed on each animal model. The liver was exposed and isolated. Multiple bleeding defects were created using a punch biopsy across the three lobes of the liver. An 8 mm biopsy punch instrument was used to create a circular defect that was approximately 2-5 mm in depth. All liver sites resulted in acceptable bleeding scores (3-4 on the WHO Bleeding Scale) following biopsy punch and prior to test article application.

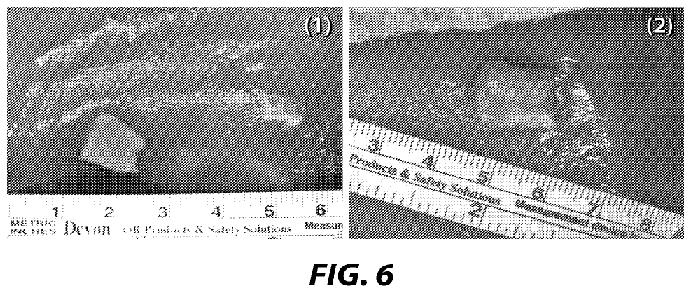

Test articles were prepared with an absorbable gelatin sponge (GelFoam®, Pfizer). The hemostatic sponge was cut into 1.5 cm×1.5 cm pieces and infused with 0.5 mL of RADA16 2.5% surgical hemostatic agent (PuraStat®). The hemostatic agent was infused by applying the PuraStat® to the sponge through a syringe and nozzle, prior to application of the sponge on the wound. Control hemostatic sponges were prepared by applying 1 mL of saline to the sponge and removing excess saline prior to application of the sponge on the wound. The prepared articles had a volume sufficient to cover the entire defect site of each wound, as shown in FIG. 6. In (1) a GelFoam® and saline test article was applied to the liver biopsy defect. In (2) a GelFoam® and PuraStat® test article was applied to the liver biopsy defect.

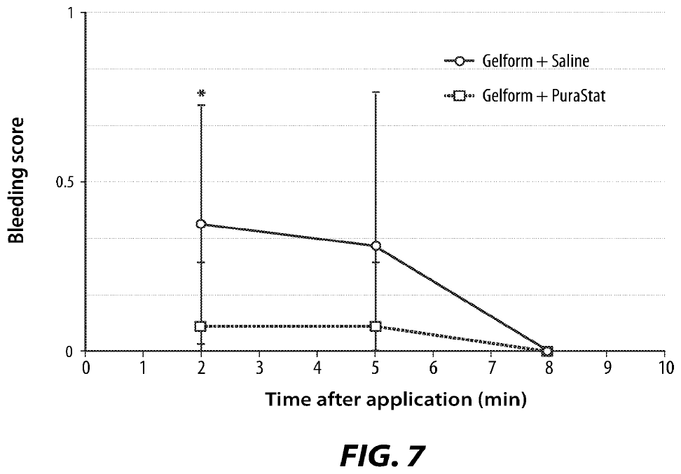

Each test article was applied to the liver wound site with pressure for approximately 2 minutes. The liver lesions were scored for bleeding immediately following the two minute pressure application period, at 5 minutes after application, and at 8 minutes after application. The results are summarized in the graph of FIG. 7. No significant difference was found for initial bleeding score (time=0) between the sites treated with GelFoam®+saline and the sites treated with GelFoam®+PuraStat®. Specifically, initial bleeding of all samples was determined to be a 3 or 4 on the WHO bleeding scale.

Bleeding was reduced in all test article preparation sites following the 2 minutes of article application with direct pressure. Test articles treated with GelFoam®+PuraStat® resulted in lower bleeding scores at 2 minutes and at 5 minutes after article application, as compared to the test articles of GelFoam®+saline. All samples exhibited no bleeding at 8 minutes after application. GelFoam®+PuraStat® hemostatic effect superiority over GelFoam®+saline hemostatic effect is especially significant at 2 minutes after application (p<0.1). Specifically, after 2 minutes, the sites treated with GelFoam®+saline exhibited an average bleeding of 0.38 on the WHO bleeding scale, while the sites treated with GelFoam®+PuraStat® exhibited an average bleeding of 0.07 on the WHO bleeding scale. The bleeding scores of GelFoam®+saline are summarized in Table 1, and the bleeding scores of GelFoam®+PuraStat® are summarized in Table 2.

TABLE 1
Bleeding Scores of GelFoam ® + saline samples.

| | Bleeding score | | | |
|---|---|---|---|---|
| | Initial bleeding | | | |
| | score before | After application | | |
| Sample # | application | 2 min | 5 min | 8 min |
| 1 | 3 | 0 | 1 | 0 |
| 2 | 4 | 1 | 1 | 0 |
| 3 | 4 | 0.5 | 0 | 0 |
| 4 | 3 | 0.5 | 0 | 0 |
| 5 | 3 | 0.5 | 0.5 | 0 |
| 6 | 3 | 0 | 0 | 0 |
| 7 | 4 | 0.5 | 0 | 0 |
| 8 | 4 | 0 | 0 | 0 |
| (Mean, SD) | (3.50, 0.53) | (0.38, 0.35) | (0.31, 0.45) | (0, 0) |

TABLE 2
Bleeding Scores of GelFoam ® + PuraStat ® samples.
Result of the t-test for two independent means between the
bleeding scores of GelFoam ® + saline and GelFoam ® + PuraStat ®

| | Bleeding score | | | |
|---|---|---|---|---|
| | Initial bleeding | | | |
| | score before | After application | | |
| Sample # | application | 2 min | 5 min | 8 min |
| 1 | 3 | 0 | 0 | 0 |
| 2 | 3 | 0 | 0 | 0 |
| 3 | 4 | 0.5 | 0 | 0 |
| 4 | 3 | 0 | 0 | 0 |
| 5 | 4 | 0 | 0.5 | 0 |
| 6 | 4 | 0 | 0 | 0 |
| 7 | 3 | 0 | 0 | 0 |
| (Mean, SD) | (3.42, 0.53) | (0.07, 0.19) | (0.07, 0.19) | (0, 0) |
| P value | 0.8003 | 0.06376 | 0.2180 | — |

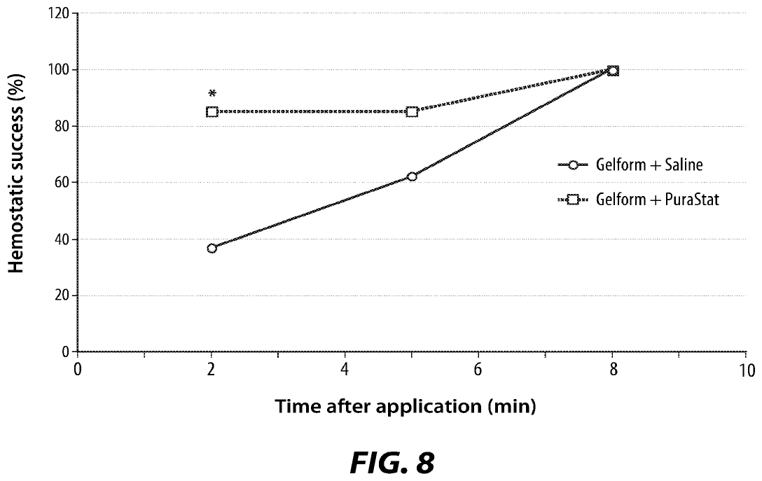

The graph of FIG. 8 shows hemostatic success (%) after application. The bleeding score of all samples after 8 minutes was 0 (100% hemostatic success). GelFoam®+PuraStat® samples showed a higher hemostatic success at 2 minutes and 5 minutes after application (87.5% and 87.5%, respectively), as compared to GelFoam®+saline (37.5% and 62.5%, respectively). Specifically, at 2 and 5 minutes after application, 6 of the 7 defect sites treated with GelFoam® and PuraStat® had achieved hemostasis, as compared to 3 of 8 defect sites that achieved hemostasis with GelFoam®+saline at 2 minutes, and 5 of 8 that achieved hemostasis with GelFoam®+saline at 5 minutes. The Z score test for two population proportions demonstrates the significant superiority of GelFoam®+PuraStat® over GelFoam®+saline. Statistical significance is found at 2 minutes after application (p<0.1). Similar results are expected with an IEIK13 1.3% (pH 3.0) self-assembling peptide hydrogel, due to the similar gelation mechanics of IEIK13 1.3% (pH 3.0) and RADA16 2.5%, as shown above in Example 3.

Accordingly, a self-assembling peptide hydrogel can be utilized with a hemostatic sponge. Furthermore, the self-assembling peptide can enhance the hemostatic efficacy of the hemostatic sponge.

It is to be appreciated that embodiments of the methods and devices discussed herein are not limited in application to the details of construction and the arrangement of components set forth in this description or illustrated in the accompanying drawings. The methods and devices are capable of implementation in other embodiments and of being practiced or of being carried out in various ways. Examples of specific implementations are provided herein for illustrative purposes only and are not intended to be limiting. Also, the phraseology and terminology used herein is for the purpose of description and should not be regarded as limiting. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present devices and methods or their components to any one positional or spatial orientation.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description is by way of example only.

1.-13. (canceled)

14. A device for hemostasis, comprising: a substantially porous wound dressing; and
a solution comprising a self-assembling peptide pervading the pores of the porous wound dressing, the self-assembling peptide comprising between about 7 amino acids and 32 amino acids in an effective amount and in an effective concentration for use in forming a hydrogel under physiological conditions to promote hemostasis.

15. The device of claim 14, wherein the solution is present in a volume of about 1 μL to 2 mL per 1 cm² of porous wound dressing.

16. The device of claim 14, wherein the wound dressing and solution capable of promoting hemostasis on a wound having an initial bleeding score of 2 or higher, as assessed on the World Health Organization (WHO) Bleeding Scale.

17. The device of claim 16, wherein the wound dressing and solution are capable of promoting hemostasis on a wound having an initial bleeding score of 3 or higher, as assessed on the WHO Bleeding Scale.

18. The device of claim 14, wherein the self-assembling peptide is selected from the group consisting of RADA16 and IEIK13.

19. The device of claim 14, wherein the self-assembling peptide comprises KLD12.

20. The device of claim 14, wherein the self-assembling peptide comprises between about 12 to about 16 amino acids that alternate between a hydrophobic amino acid and a hydrophilic amino acid.

23. The kit of claim 14, wherein the wound dressing comprises cotton gauze.

21. The device of claim 14, wherein the solution is substantially free of cells and/or drugs.

22. The device of claim 14, wherein the wound dressing comprises a sponge, a woven textile, a non-woven textile, a puff, or a mixture thereof.
